(19) United States
(12) Patent Application Publication
(10) Pub. No.: US 2014/0222125 A1
(43) Pub. Date: Aug. 7, 2014

(54) Intravascular Electrodes for Transvenous Stimulation

(71) Applicant: Synecor LLC, Chapel Hill, NC (US)

(72) Inventors: Richard A. Glenn, Chapel Hill, NC (US); Jeffrey A. Smith, Petaluma, CA (US); Geoffrey A. Orth, Sebastopol, CA (US); Michael S. Williams, Santa Rosa, CA (US)

(73) Assignee: Synecor LLC, Chapel Hill, NC (US)

(21) Appl. No.: 14/105,152

(22) Filed: Dec. 12, 2013

Related U.S. Application Data
(63) Continuation of application No. 13/068,866, filed on Jul. 11, 2011, now abandoned.
(60) Provisional application No. 61/378,925, filed on Aug. 31, 2010.

(51) Int. Cl. A61N 1/05

(52) U.S. Cl. CPC A61N 1/05
USPC 607/116

(57) ABSTRACT

An intravascular electrode device for use in neuromodulation includes an anchor expandable from a radially compressed position to a radially expanded position. A lead extends from the anchor and has at least one conductor extending through it. A flex circuit is coupled to the anchor and comprises a flexible insulative substrate, a plurality of electrodes carried by the substrate, and a plurality of conductive traces carried by the substrate, each trace electrically coupled to an electrode and a conductor. Expansion of the anchor within a blood vessel biases the electrodes into contact with the surrounding blood vessel wall.

This application is a continuation of co-pending U.S. Ser. No. 13/068,866, filed Jul. 11, 2011, which claims the benefit of U.S. Provisional Application No. 61/378,925, filed Aug. 31, 2010.

### TECHNICAL FIELD OF THE INVENTION

The present application generally relates to intravascular electrodes and associated methods for delivering therapy to nervous system targets.

### BACKGROUND

Applicant's prior Application Publication No. U.S. 2007/0255379, discloses an intravascular neurostimulation device (such as a pulse generator) and associated methods for using the neurostimulation device to stimulate nervous system targets. In various ones of the disclosed embodiments, electrodes positioned within a blood vessel (e.g. a jugular vein, superior vena cava, or inferior vena cava) are used to transvascularly stimulate nervous system targets located outside the vasculature. Such stimulation can be used to lower heart rate and/or control blood pressure as a treatment for hypertension or HF. Anchors are described for maintaining the electrodes in contact with the blood vessel wall. The anchors include structural features that allow the anchor to radially engage a vessel wall. As described, a band, sleeve, mesh or other framework formed of one or more shape memory (e.g. nickel titanium alloy, nitinol, thermally activated shape-memory material, or shape memory polymer) elements or stainless steel, Elgiloy, or MP35N elements may be used as an anchor. In use, the anchor (with the electrodes thereon) may be released from a sheath within the blood vessel, such that the anchor expands into contact with the blood vessel and thereby biases the electrodes against the vessel wall.

Applicant's co-pending application Ser. No. 12/413,495, filed Mar. 27, 2009 and entitled SYSTEM AND METHOD FOR TRANS VASCULARLY STIMULATING CONTENTS OF THE CAROTID SHEATH discloses a method for transvascularly stimulating the vagus nerve and other nervous system structures, such as those disposed within the carotid sheath. The disclosed method includes advancing an energy delivery element, which may be an electrode, into an internal jugular vein, retaining the energy delivery element in a portion of the internal jugular vein contained within a carotid sheath, and energizing the energy delivery element to transvenously direct energy to target contents of the carotid sheath external to the internal jugular vein. The energy may be directed to a carotid artery within the carotid sinus sheath, and/or to a carotid sinus nerve or nerve branch within the carotid sinus sheath, to nerve branches emanating from carotid artery baroreceptors, and/or to a vagus nerve or associated nerve branch within the carotid sinus sheath. In some of the disclosed embodiments, a bi-lateral system is employed, in which a second electrode or other second energy delivery element is introduced into a second internal jugular vein and retained in a portion of the second internal jugular vein contained within a second carotid sheath. The second energy delivery element is energized to direct energy to contents of the second carotid sheath external to the second internal jugular vein. The right vagus nerve primarily innervates the sinoatrial node of the heart;

stimulation of this nerve increases the duration of the cardiac cycle. The left vagus nerve primarily innervates the atrioventricular (AV) node of the heart; stimulation of this nerve slows AV conduction. The assignor of the present application conducted anatomical studies on human cadavers to investigate the relative location of the right vagus nerve to veins that could provide sites for transvenous vagal stimulation to reduce heart rate and blood pressure. The findings strongly support the rationale for a transvenous approach to vagus nerve stimulation in the human. The right vagus nerve and its cardiac branches closely and reliably course directly alongside the largest veins in the neck and superior mediastinum, namely the right internal jugular vein, right brachiocephalic vein, superior vena cava, and azygotic arch.

### BRIEF DESCRIPTION OF THE DRAWINGS

FIG. 1A is a perspective view showing a first embodiment of an electrode array together with a temporary anchor for use during mapping.

FIG. 1B is a side elevation view showing the array and anchor of FIG. 1A schematically disposed within a blood vessel.

FIG. 2A is similar to FIG. 1A but shows the anchor and array separated from one another.

FIGS. 2B-2D show the array of FIG. 2A with alternate temporary anchor designs.

FIG. 3 is a distal end view of the FIG. 1A embodiment.

FIG. 4 is a side elevation view of the FIG. 1A embodiment.

FIG. 5 is a perspective view showing the FIG. 1A array using the more permanent anchor.

FIG. 6 is an exploded view of the array and permanent anchor of FIG. 5.

FIGS. 7 and 8 are perspective views of a second embodiment of an array and anchor.

FIG. 9 is an exploded view of the system of FIG. 7.

FIG. 10 is a distal end view of the array of FIG. 7 and shows the anchor exploded from the array.

FIG. 11 shows an alternative embodiment of an array.

FIG. 12 schematically shows the FIG. 11 embodiment in a curled position within a vessel.

FIG. 13 is similar to FIG. 12 but does not show the vessel.

FIG. 14 illustrates an alternative embodiment of an anchor for retaining an electrode array within a blood vessel.

FIGS. 15 through 20 show exemplary data generated during use of an electrode of the type disclosed herein.

### DETAILED DESCRIPTION

The present application describes designs of intravascular electrodes that may be positioned within the vasculature and used for stimulation of nervous targets.

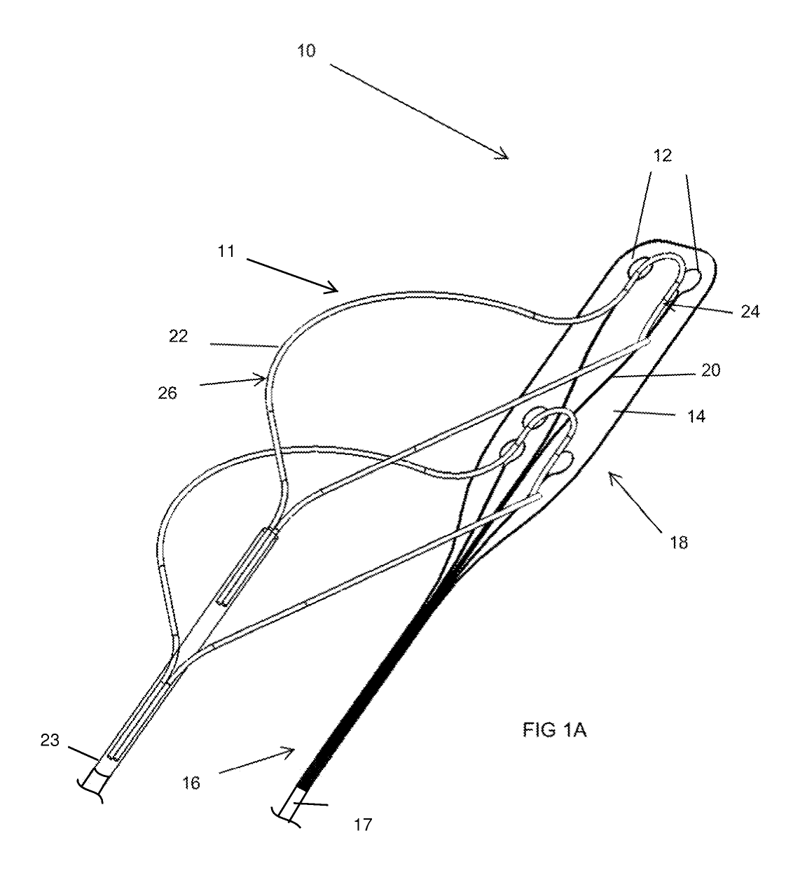

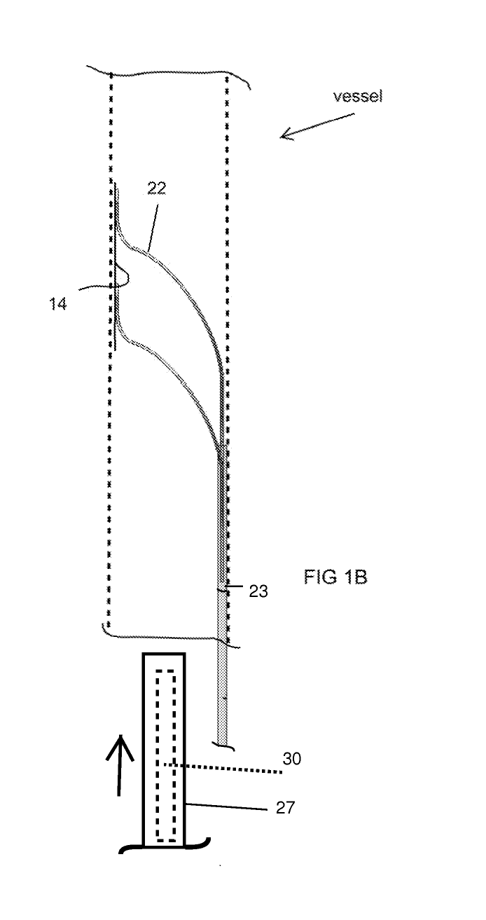

FIG. 1A illustrates a first embodiment of an electrode array 10 that is positionable within a blood vessel (such as the vessels discussed herein) and coupled to an intravascular, subcutaneous, or extracorporeal pulse generator (not shown) for use in transvascular stimulation. The array allows for programmable modulation of electrode selection in response to position-mediated signal adaptation. Array 10 comprises a plurality of electrodes 12 positioned on a flexible substrate 14. Four electrodes are shown in a 2×2 array, although various electrode numbers and arrangements may be used. The electrodes are positioned on the face of the substrate that faces the vessel wall when the system is implanted, thus placing the active electrode surfaces in contact with the vessel wall.

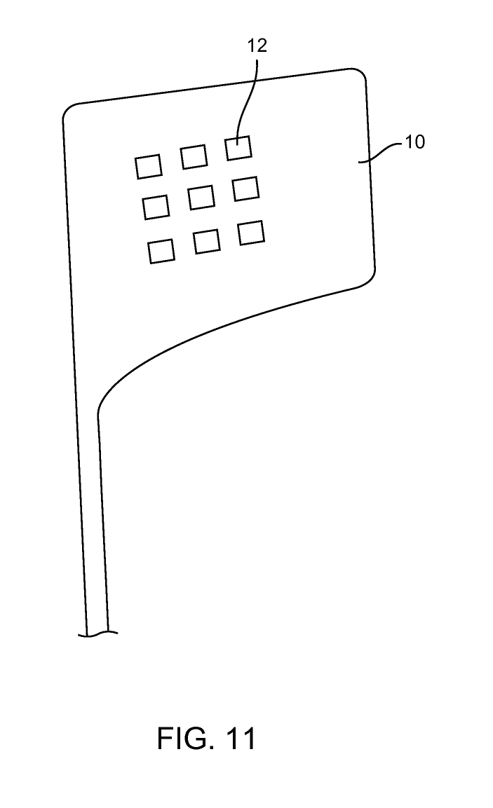

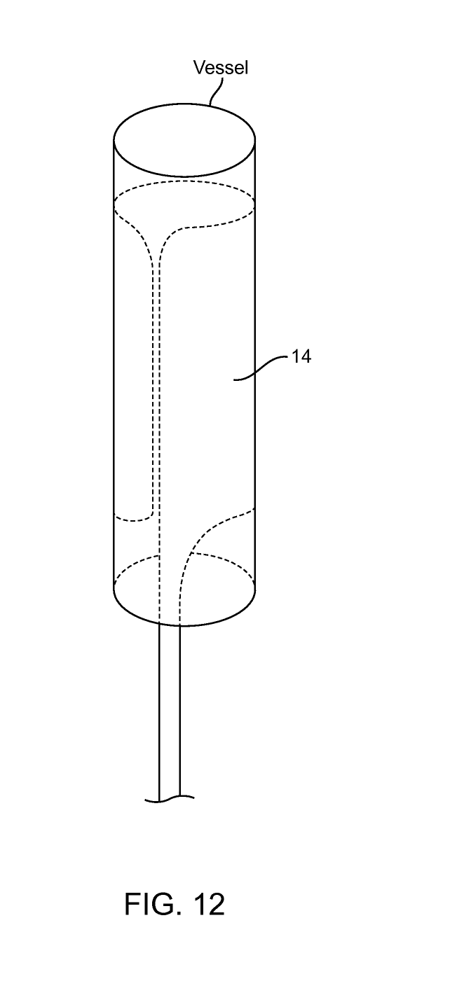

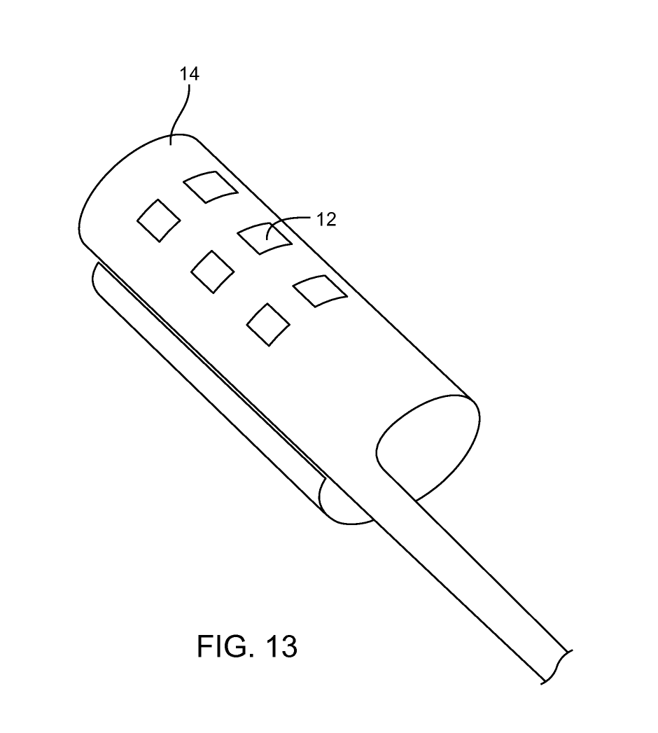

The substrate includes a relatively narrow portion 16, and a broader paddle portion 18 on which the electrodes are positioned. Although the longitudinal axes of the narrow and broader portions 18 are longitudinally aligned, in alternative embodiments the paddle portion 18 may be positioned asymmetrically relative to the longitudinal axis of the narrow portion. Embodiments of this type are illustrated in FIGS. 11-12B.

The electrodes are deposited on one face of the substrate 14, such that the substrate provides an electrically insulative backing and electrically isolates the electrodes from one another. In other embodiments, the electrodes may be positioned in openings formed through the substrate. Conductive traces 20 are formed on the substrate and extend proximally from each of the electrodes 12, terminating at contacts near the proximal end of the narrow portion 16. A lead 17 which may be formed of tubing, shrink material, or other suitable material, is disposed over at least a portion of narrow portion 16, and includes conductors electrically coupled to the contacts of the traces 20.

The substrate is preferably a material that provides an electrically insulative backing to the electrodes. The material might be one capable of curving relative to the vessel's longitudinal axis to approximately match the curvature of a blood vessel wall when held in contact with the wall by an anchor (see e.g. the FIG. 5 anchor). The substrate will similarly be placed in a curved position when disposed within a delivery sheath for introduction into the vasculature. The substrate 14 may be a flex circuit formed of polyimide or other suitable materials. Alternate materials that may be used for the substrate include, but are not limited to, polyurethane, silicone rubber, fluoropolymer, stainless steel, platinum-iridium, MP35N, titanium and other biocompatible metals/polymers/elastomers.

In some embodiments, all or a portion of the substrate may be of a type that resorbs or degrades over time, as tissue growth (e.g., cellular encapsulation, in-growth, endothelialization) begins to retain the electrodes in position. Materials suitable for this use include, but are not limited to, polylactide (PLA), polyglycolide (PGA) and their copolymer (PGLA). In such embodiments, the electrodes may be provided with non-degradable insulating material on the portions of the electrodes which are not intended for contact with the vessel wall, such that the insulating material remains intact following resorption or degradation of the substrate. In other embodiments, the flex circuit may be coated to improve its biocompatibiliy and to reduce the body's response to a foreign substance.

For array implantation, it is desirable for the user to be able to empirically select an electrode location by positioning the electrode array, delivering stimulation from the selected location, measuring the response, and then repeating the process with the electrodes at one or more different locations within the blood vessel. This mapping process allows the user to evaluate the response at various stimulation sites, so s/he may select the most optimal stimulation site for more permanent array positioning.

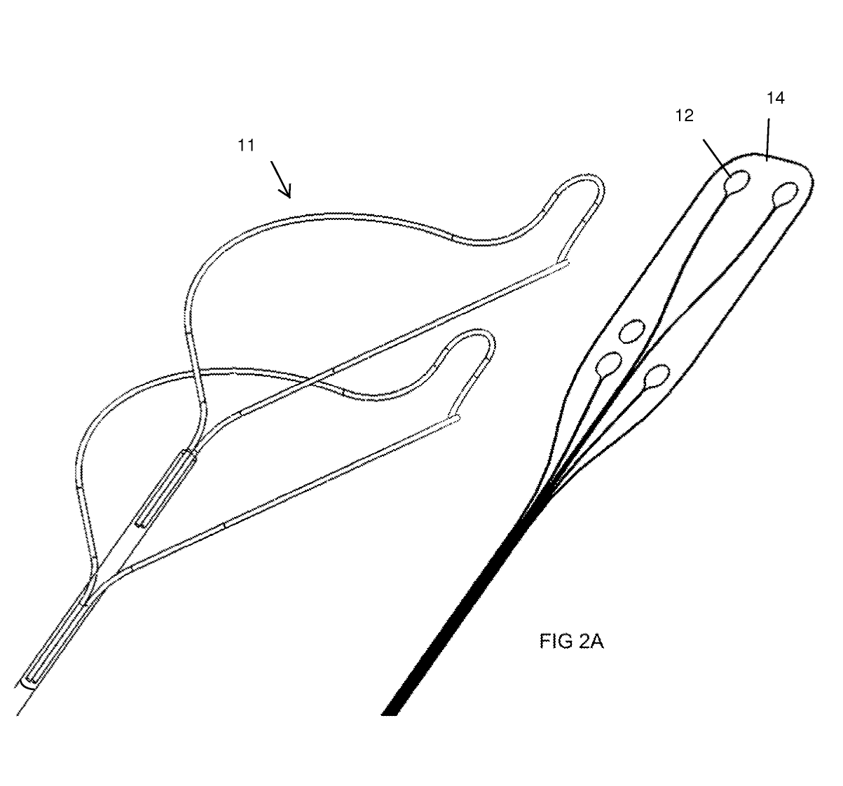

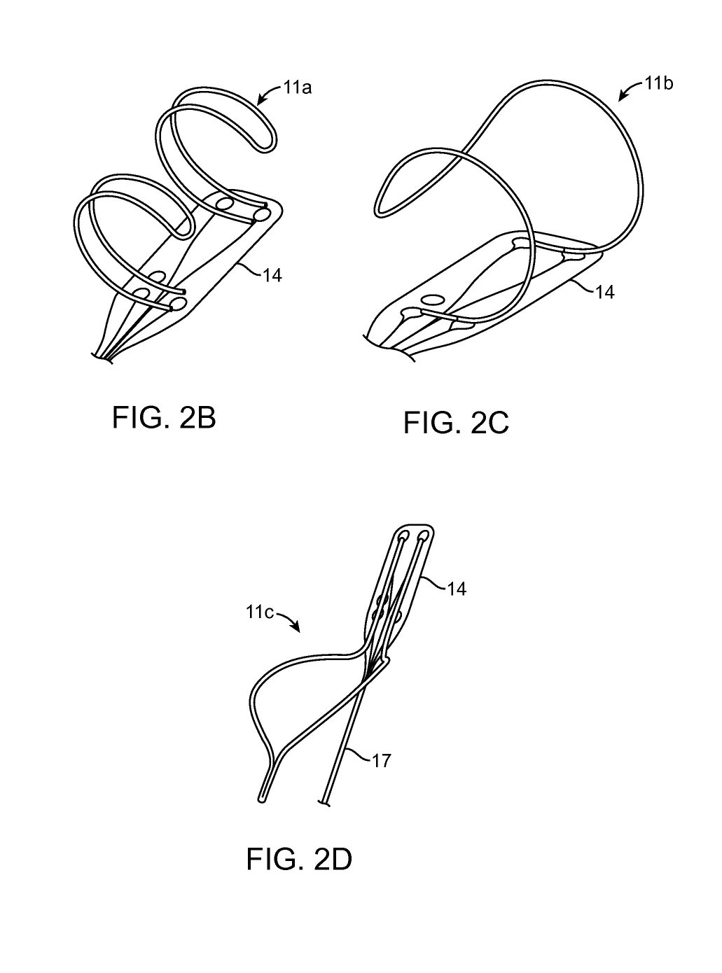

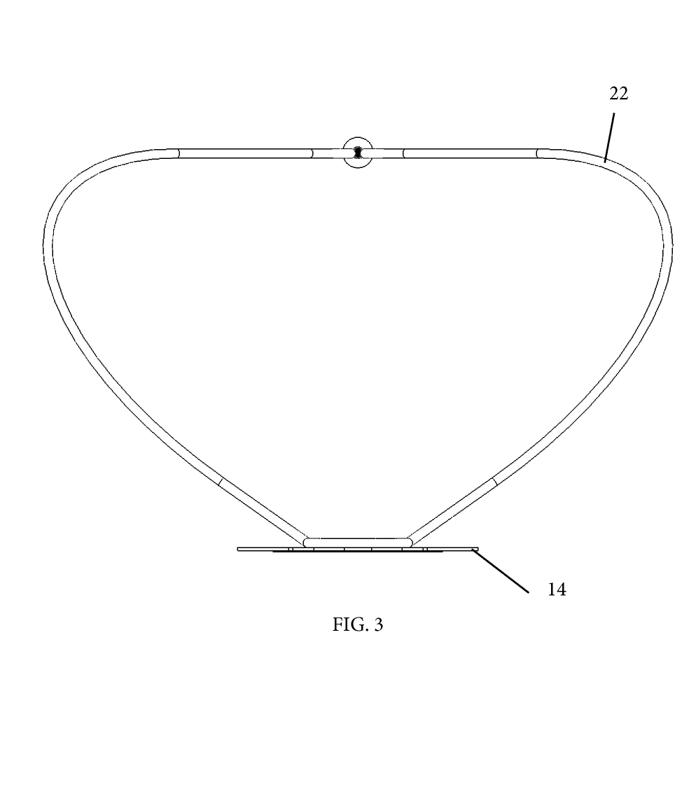

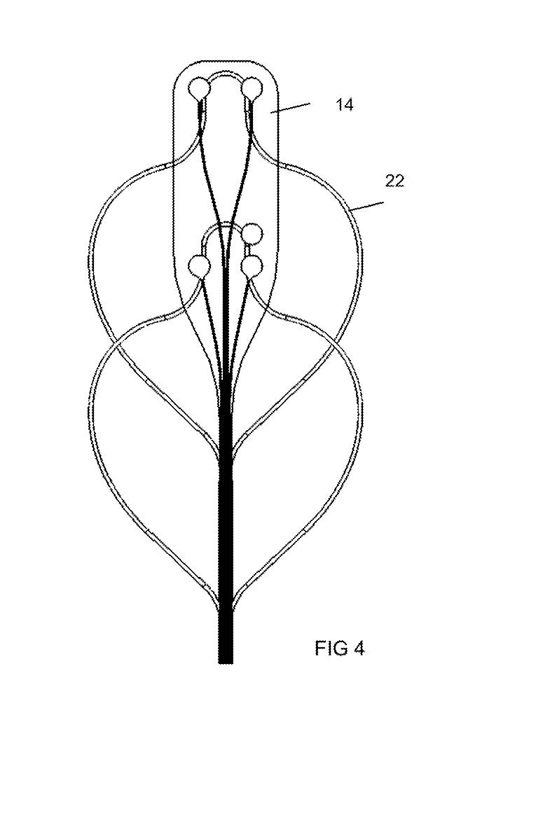

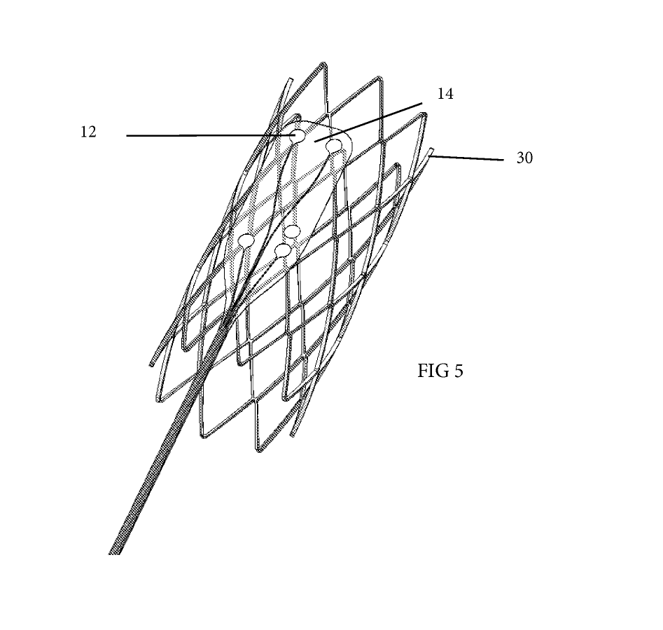

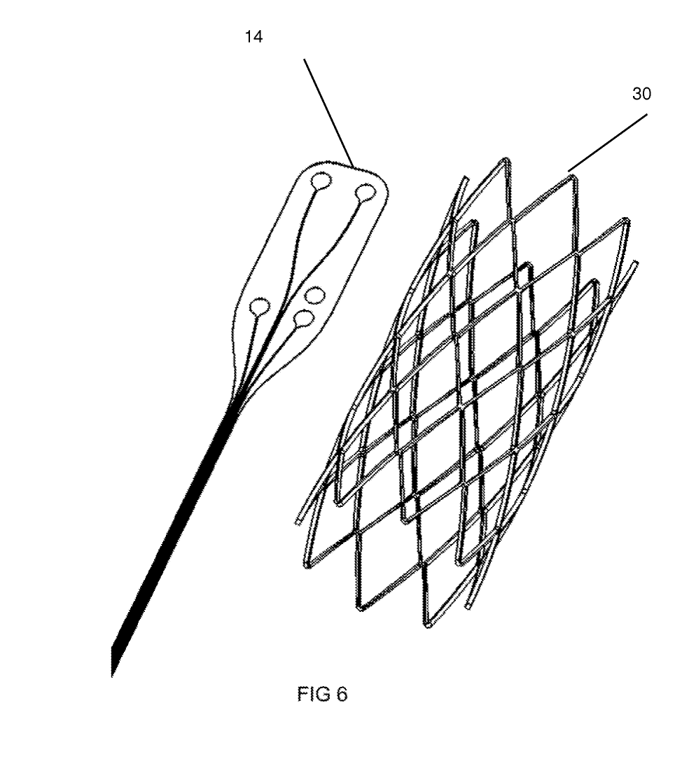

In the first embodiment, a temporary anchor 11 is positionable in contact with the substrate 14 for use in retaining the array during mapping. The temporary anchor 11 may be releasably attached to the substrate 14, or it may be separate from the substrate. Referring to FIG. 1B, the temporary anchor 11 is formed of one or more nitinol loops 22 positioned on an elongate shaft 23. Each loop has a substrate contacting portion 24 and a vessel wall contacting portion 26 that extends away from the portion 24. The vessel wall contacting portion 26 is shaped to contact the vessel wall at one or more points so as to bias the electrodes against the vessel wall as shown in FIG. 1B. In one example shown in FIGS. 1-2A, the substrate contacting portion 24 may be a u-shaped tip portion defined by generally parallel wire sections occupying a plane.

Other temporary anchor shapes include, but are not limited to, those shown in FIGS. 2B-2D. In the FIG. 2B embodiment, two anchors 11 a are shown, each of which comprises a length of wire formed into a “U” shaped element. The wire ends forming the legs of the “U” are coupled to the substrate 14, spaced from one another (and from the legs of the other of the anchors) in a longitudinal direction relative to the substrate. The curved base of the “U” forms a free end of the anchor. The portion of each anchor extending from the legs to the base of the U curves around the longitudinal axis of the substrate so as to allow the anchor to circumferentially contact the vessel wall when deployed.

The FIG. 2C anchor 11b is similar to that of FIG. 2B but uses a single wire positioned with the legs of the “U” further apart along the longitudinal length of the substrate 14.

The FIG. 2D anchor 11c is similar to that of FIG. 2A, but uses a single anchor loop.

The FIG. 1-2C embodiments further include a second anchor 30 provided for more permanently anchoring the array within the blood vessel once the optimal array position has been selected. As shown in FIG. 5, the second anchor 30 may be an expandable nitinol sleeve or stent-like device. It may be advanced from a catheter 27 passed through the loops 22 of the temporary sleeve as shown in FIG. 1B. The anchor expands as it is advanced from the catheter, thus sandwiching the array between the anchor 30 and the vessel wall.

In use, the array and temporary anchor are disposed within a delivery sheath (not shown). The sheath is advanced to a desired location with a target vessel (e.g. the superior vena cava for vagus nerve stimulation). The array and temporary anchor are released from the sheath. Mapping is achieved by releasing and engaging the electrode against the vessel wall, stimulating and observing the response, then, if necessary, recovering the array and anchor into the sheath. The sheath is advanced to another location and the process is repeated until the target location (at which the most optimal response to stimulation is measured) is identified.

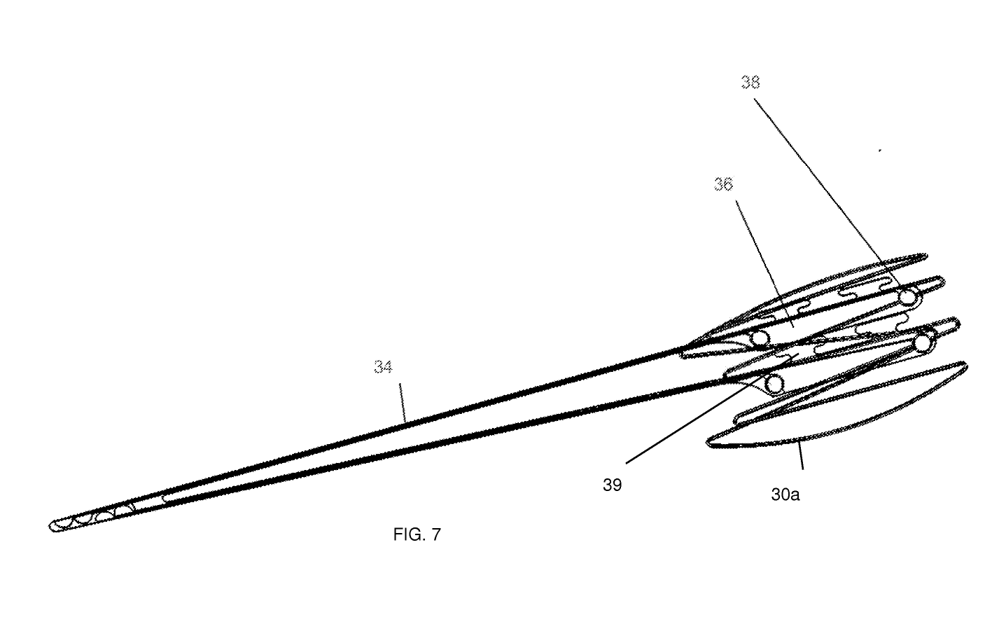

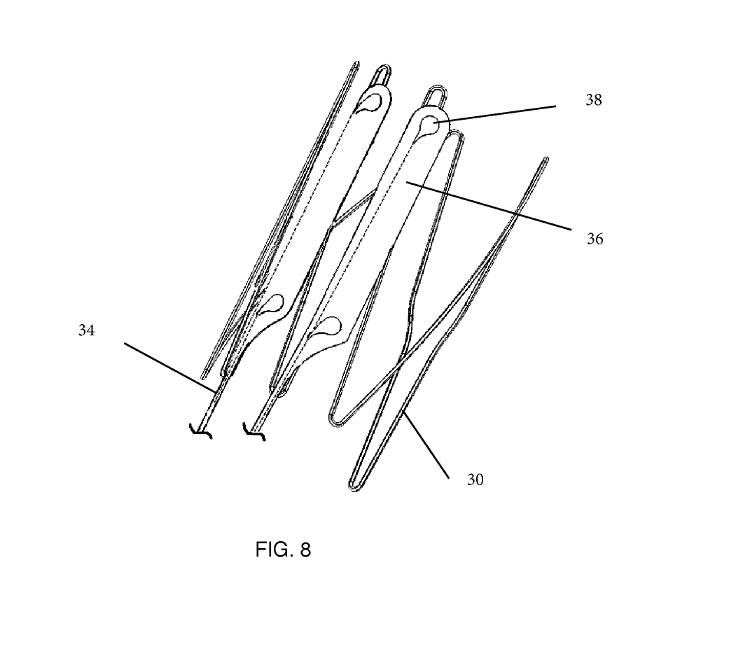

A second array system shown in FIG. 7 utilizes a self-expanding anchor 30a in combination with a flexible electrode array 32, which may be attached to the anchor or which may simply be positioned in contact with the anchor. The array includes two or more longitudinal splines 34, each supporting a flexible circuit arms 36. The arms 36, and optionally the splines 34, may be formed of flexible materials of the type disclosed above in connection with FIG. 1A.

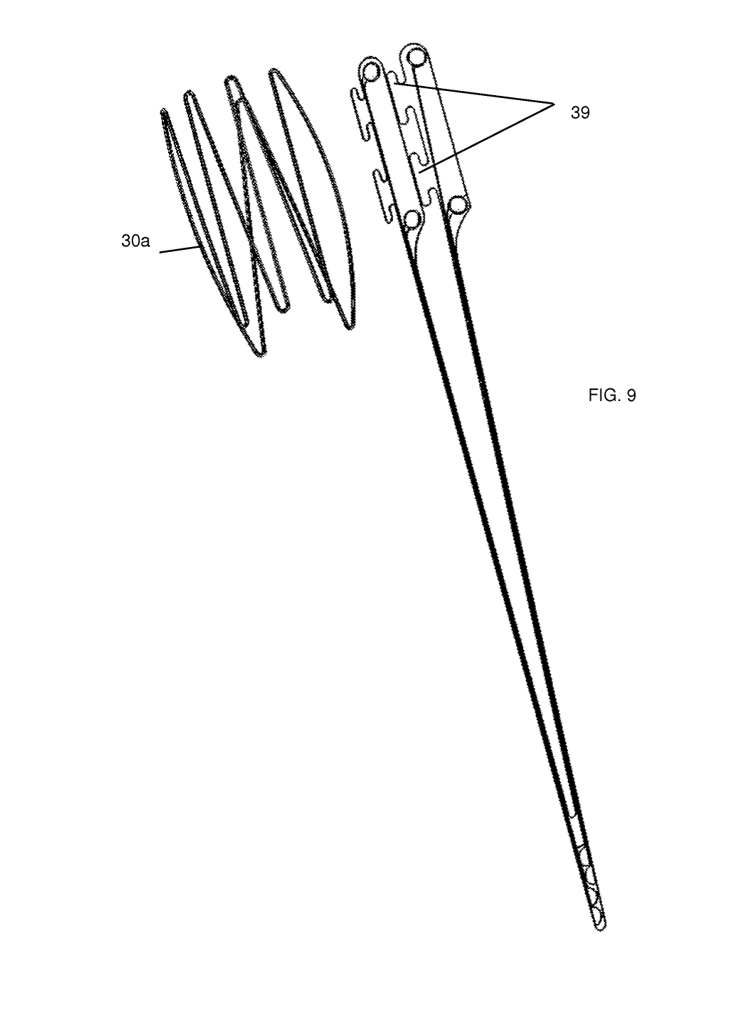

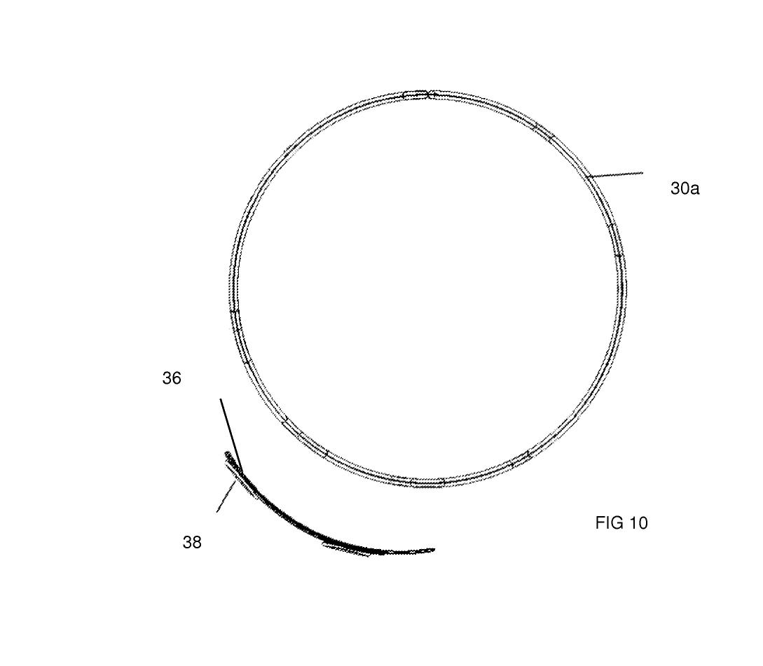

The flexible substrate material of the array 32 may include a plurality of tabs 39. The tabs are most easily seen in FIG. 9. During manufacture, tabs 39 are folded around struts of the anchor 30a and secured. FIG. 7 shows the assembly before the tabs 39 have been folded over the struts of the anchor. FIG. 8 shows the assembly after the tabs have been folded.

Two or more electrodes 38 are longitudinally arranged on each arm 36. When the system is assembled, the electrodes 38 are positioned such that their conductive surfaces face away from the anchor 30a as shown, and so that they will contact the inner wall of the target vessel when the anchor 30a is expanded. See FIG. 8.

As with the first embodiment, conductive traces are formed on the substrate and extend proximally from each of the electrodes, terminating at contacts near the proximal end of the narrow portion splines. A lead (not shown) which may be formed of tubing, shrink material, or other suitable material, is disposed over at least a proximal portion of the array (such as where the splines meet at the proximal end), and includes conductors electrically coupled to the contacts of the traces.

The second array system is deployable from a sheath as discussed above. After mapping is completed using a process similar to that described above, the anchor-electrode assembly is expanded and firmly deployed against the target vessel, maintaining the mapping-defined orientation.

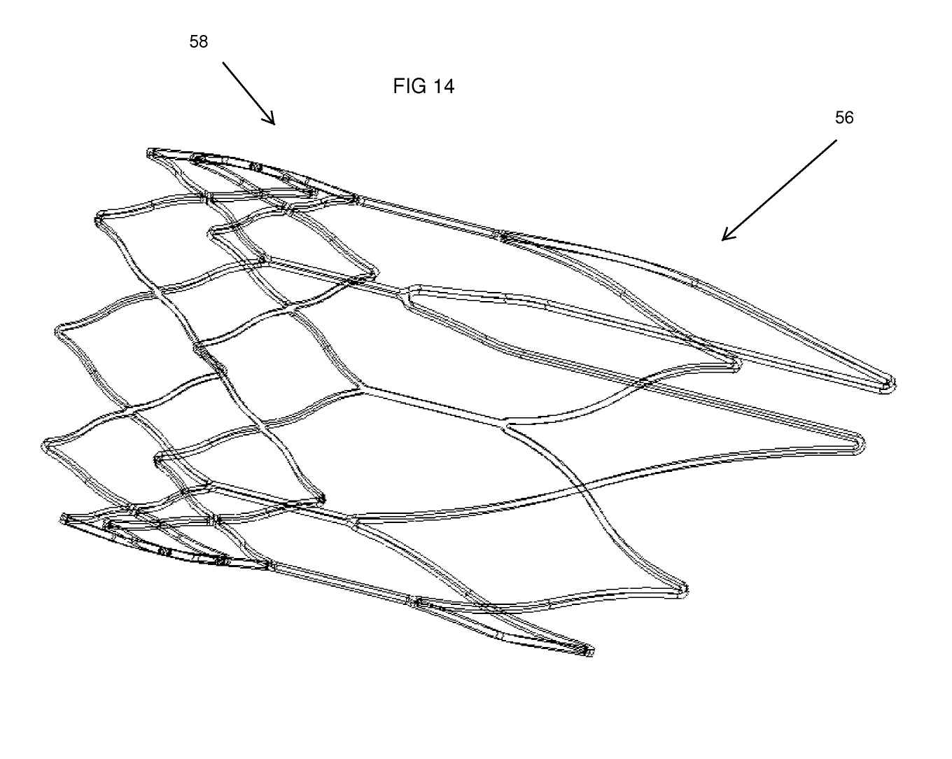

FIG. 14 illustrates an alternative anchor in which both the temporary anchor (used to temporary hold the electrodes in contact with the vessel wall during mapping), and the more permanent anchor (which is deploy after mapping has been completed to firmly anchor the electrodes within the vessel) are integrated into a single structure. Electrodes of the type disclosed herein, or alternative forms of electrodes, are mounted to or formed on the anchor so as to contact the surrounding vessel wall when the anchor is expanded.

The anchor may be actively expandable but is more preferably self-expandable when released from a sheath. The anchor includes at least a first portion 56 configured to temporarily retain the electrodes in position against the vessel wall for mapping, and a second portion 58 that will chronically retain the implant at the chosen position within the vessel once the optimal array position has been selected. In a preferred embodiment, the anchor is an expandable stent-like sleeve, and the first (temporary) anchor portion 56 is positioned distally of the second (chronic) anchor portion 58. The first and second portions are configured such that the radial expansion forces of the second portion are greater than the radial expansion forces of the first portion.

In use, the integrated anchor is disposed within a delivery sheath. The sheath is advanced to a desired location with a target vessel (e.g. the superior vena cava for vagus nerve stimulation). The first (temporary) portion 56 of the anchor is released from the sheath, placing the electrodes into contact with the vessel wall. Mapping is performed at the target location. The properties of the first portion allow it to be resheathed, and then repositioned and redeployed so that mapping may be carried out at additional sites if necessary. Once the optimal stimulation site is determined, the sheath is fully withdrawn to release the second (chronic) portion 58 of the anchor, thus firmly anchoring the electrodes at the chosen location within the blood vessel.

The disclosed electrodes may be utilized for transvenous electrical stimulation from within the superior vena cava (SVC) or internal jugular vein to the vagus nerve to achieve reduction in blood pressure and heart rate, such as for treatment of congestive heart failure or other conditions.

### Examples

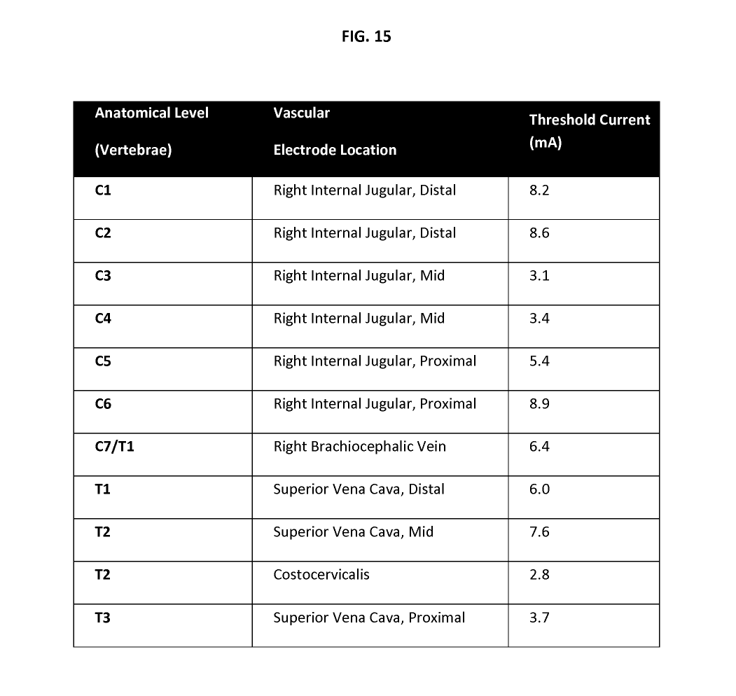

Animal studies were conducted in an effort to characterize parameters for electrical stimulation of the vagus nerve and non-target tissues. Results are presented in the graphs at FIGS. 15-29. In animal models, multipolar electrode catheters were positioned at standard fluoroscopic sites within the right internal jugular vein, right brachiocephalic vein, right costocervicalis vein, and the SVC and used to stimulate the adjacent right vagus nerve and its cardiac branches through the vein wall.

Transvenous vagal neurostimulation with appropriate parameters and orientation reliably and reproducibly achieved Significant Cardiovascular Effect (defined as concurrent decrease in mean arterial pressure (MAP) >10% and increase in R-R interval (duration of cardiac cycle) >20%) at intravascular sites corresponding to the level of each cervical vertebrae C1-C7 and thoracic vertebrae T1-T3.

Threshold testing was conducted at each anatomic level from C1-T3 and systematic stimulus-response testing was performed for each of 3 key variables: intensity (current), frequency, and duration (pulse width).

Appropriate parameter selection enabled sustained significant cardiovascular effect up to 12 minutes (longest tested duration) without aberrant oscillation of heart rate and blood pressure; hemodynamic parameters typically returned to baseline within 30 seconds after stimulation without rebound tachycardia.

In no case did an animal become permanently refractory to vagal stimulation after significant cardiovascular effect was achieved, although vagal overdrive with supramaximal stimulation parameters transiently (<120 seconds) increased the stimulation threshold for significant cardiovascular effect in some cases.

Muscarinic blockade by administration of atropine abolished the cardiovascular effects of vagal nerve stimulation, indicating that these effects are mediated by efferent vagal parasympathetic fibers.

Collateral stimulation of non-target tissue predictably varied with stimulation location based on regional anatomy. Appropriate selection of stimulation parameters enabled achievement of significant cardiovascular effect without evidence of adverse effects (stimulation of other regional nerves or muscles) at all levels with the exception of C1 and C3, where stimulation invariably caused significant vibrations in the cervical musculature.

Stimulus response testing was conducted in canine models. Significant cardiovascular effects (reduction in MAP >10% increase in R-R interval >20%) were achieved with transvenous vagal nerve stimulation at each anatomic level from C1-T3 at threshold currents ranging from 2.8-8.9 mA. Stimulation sites with threshold current requirements ≦6 mA were identified in each of the following vessels: right internal jugular vein, right brachiocephalic vein, right costocervicalis vein, and SVC. The lowest thresholds identified were at C3 (3.1 mA), C4 (3.4 mA), T3 (3.7 mA), and in the costocervicalis vein at the level of T2 (2.8 mA).

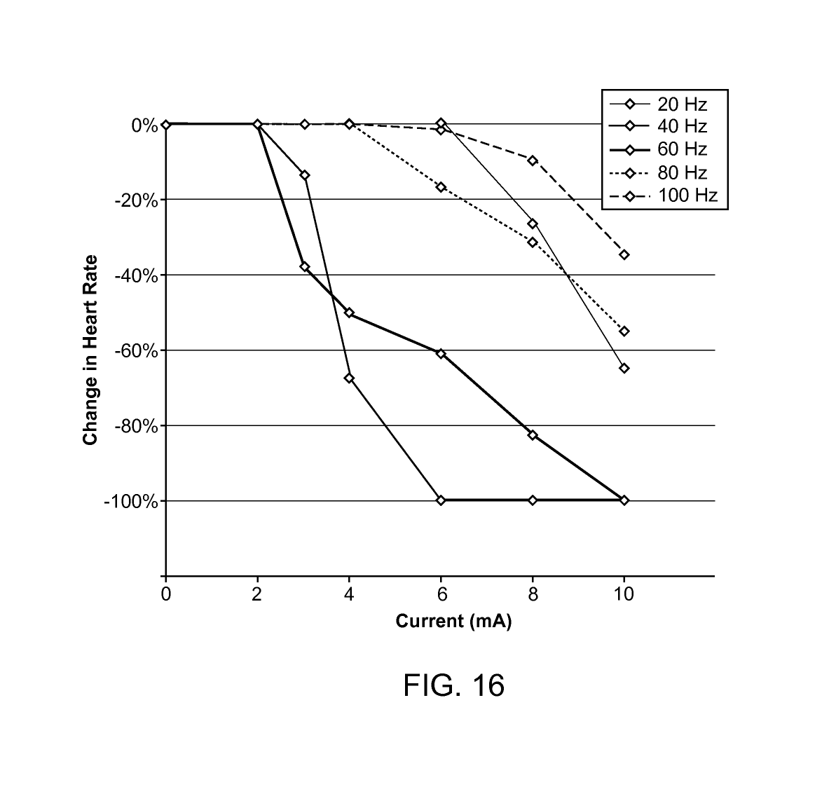

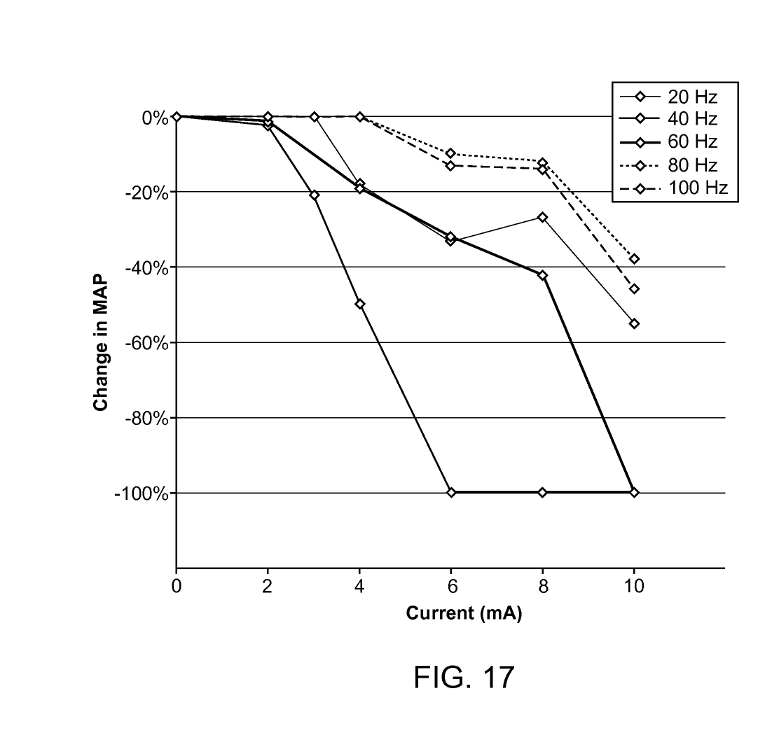

Increasing current above threshold produced an amplitude-dependent increase in cardiovascular effect. Strength-response curves are presented in Graphs 2 and 3 (FIGS. 16-17). In the most effective frequency range (20-40 Hz), stimulation with supramaximal intensity reliably produced asystole that persisted for the duration of stimulation and spontaneously resolved upon its cessation. For every set of stimulation parameters that produced asystole, reduction in current intensity produced bradycardia instead.

Amplitude in the present study is primarily represented in terms of current (mA) rather than voltage (V) in order to facilitate direct comparison of the extracellular electric field generated by different multipolar electrode types with different average impedances at the electrode-tissue interface. The average impedances for the first electrode catheter (used here for transvenous vagal stimulation from the internal jugular vein) and the second electrode catheter (used for transvenous vagal stimulation from the SVC) were 730 Ω and 1150 Ω, respectively. For considerations of power consumption, it should be noted that significant cardiovascular effect was achieved at every level from C1-T3 at output voltages ranging from 2.5V-8.8V (mean 4.8V). For reference, currently-available voltage-regulated pulse generators for neurostimulation are programmable to deliver voltage up to 10.5V.

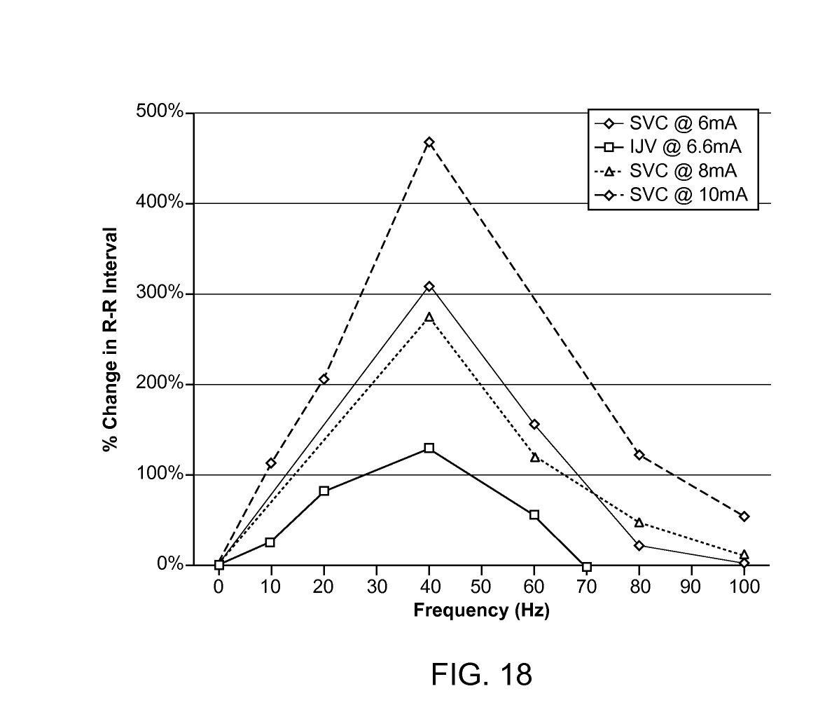

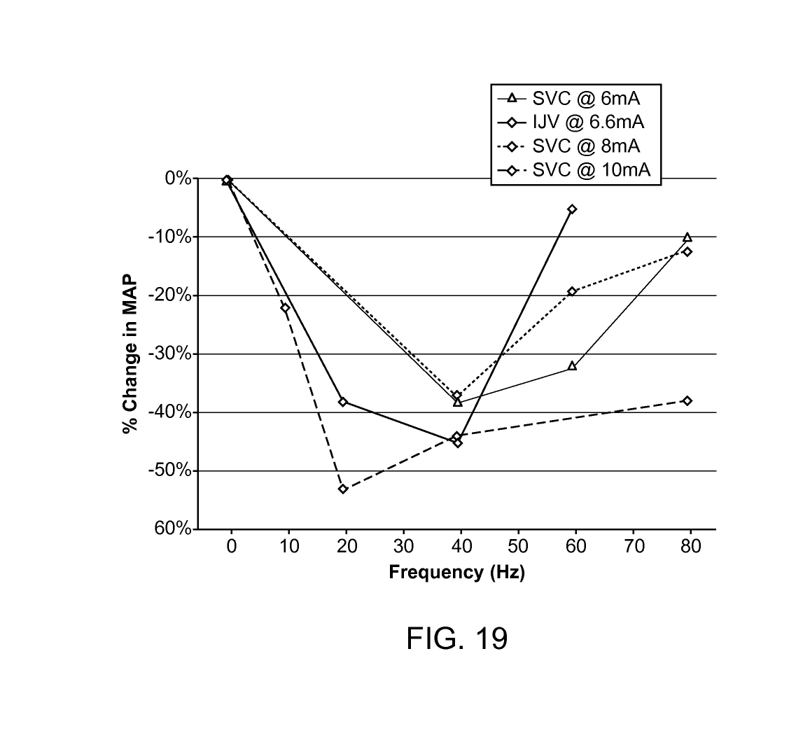

Frequency-response testing at constant suprathreshold current and duration demonstrated progressive slowing of the cardiac cycle with increasing stimulation frequency up to 40 Hz. Response progressively deteriorated as frequency was further increased above 40 Hz (up to 100 Hz), presumably due to neural fatigue. Similarly, increasing frequency causes a progressive drop in MAP up to 20-40 Hz; further increasing frequency beyond this optimal range results in suboptimal MAP response. Frequency-response curves for transvenous vagal nerve stimulation are presented in Graphs 4 and 5 (FIGS. 18-19). Overall, frequencies between 10-40 Hz were found to be most reliable and effective for achieving significant cardiovascular effects using transvenous nerve stimulation. These findings are consistent with published reports of optimal stimulation frequencies for direct vagal nerve stimulation for heart rate reduction.

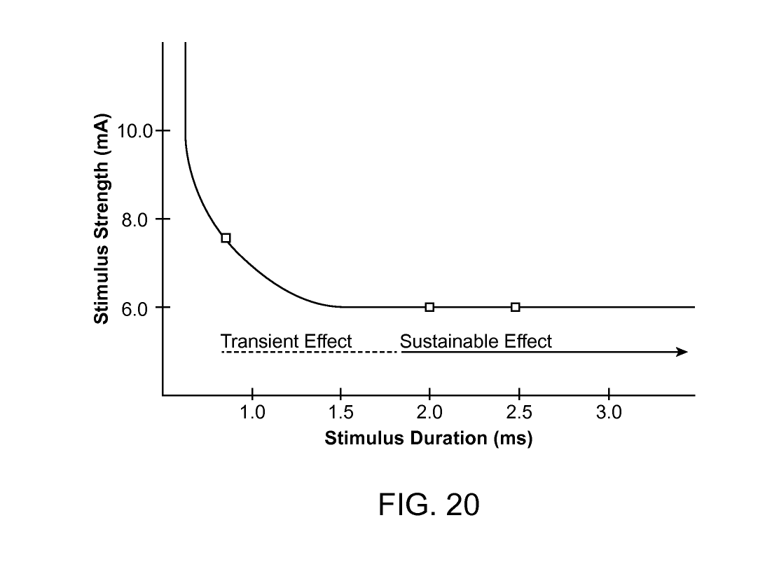

Strength-duration testing demonstrated reliable, reproducible cardiovascular effects of transvenous vagal nerve stimulation for pulse widths >2 ms (Graph 6, FIG. 20). Increasing pulse width >2.5 ms did not reduce threshold current for significant cardiovascular effect. Strength-duration curves for effects on MAP and R-R interval did not differ. Stimulation with pulse widths shorter than 1.8 ms routinely produced only transient, blunted cardiovascular responses. Capture was rarely observed for pulse widths <0.5 ms.

All prior patents and applications referred to herein, including for purposes of priority, are incorporated by reference for all purposes. It should be recognized that a number of variations of the above-identified embodiments will be obvious to one of ordinary skill in the art in view of the foregoing description. Accordingly, the invention is not to be limited by those specific embodiments and methods of the present invention shown and described herein. Rather, the scope of the invention is to be defined by the following claims and their equivalents.

What is claimed is:

1. An intravascular electrode device, comprising:
an anchor having a first, radially compressed, position and a second, radially expanded, position;
a lead extending from the anchor and having at least one conductor extending therethrough; and
a flex circuit coupled to the anchor, the flex circuit comprising a flexible insulative substrate, a plurality of electrodes carried by the substrate, and a plurality of conductive traces carried by the substrate, each trace electrically coupled to an electrode and a conductor.

2. The intravascular electrode device of claim 1, wherein the anchor comprises a generally tubular structure having a lumen.

3. The intravascular electrode device of claim 2, wherein the anchor is a first anchor and the device further includes a second anchor, the second anchor insertable in a compressed position into the lumen of the first anchor when the first anchor is in the expanded position within a blood vessel, and wherein the second anchor is expandable within the lumen to an expanded position to chronically retain the electrodes in contact with a wall of the blood vessel.

4. The intravascular device of claim 1, wherein the flex circuit comprises a pair of elongate flex circuit elements longitudinally arranged on the anchor, each flex circuit element having at least two electrodes.

5. The intravascular device of claim 4, wherein the flex circuit further includes flex circuit tail sections extending proximally from the elongate flex circuit elements to the lead, each flex circuit tail having conductive traces thereon.

6. The intravascular device of claim 5, wherein the flex circuit elements and the flex circuit tail sections each have widths in a direction orthogonal to the longitudinal direction, and wherein the width of each flex circuit tail section is less than the width of its corresponding flex circuit element.

7. The intravascular device of claim 1 wherein the substrate comprises polyimide.

8. The intravascular device of claim 1, further including a coating on at least a portion of the polyimide.

9. The intravascular device of claim 1, wherein the substrate comprises polyurethane.

10. The intravascular device of claim 1, wherein the substrate has a first surface and a second surface opposite to the first surface, the first surface positioned radially outwardly when the anchor is in the expanded position, wherein each electrode has an exposed conductive surface at the first surface of the substrate.

11. A method of anchoring an electrode array within a blood vessel, comprising the steps of:
providing an implant comprising an anchor, a lead extending from the anchor and having at least one conductor extending therethrough, and a flex circuit coupled to the anchor, the flex circuit comprising a flexible insulative substrate, a plurality of electrodes carried by the substrate, and a plurality of conductive traces carried by the substrate, each trace electrically coupled to an electrode and a conductor;
with the anchor in a radially compressed position, percutaneously introducing the implant into a patient's vasculature;
positioning the implant at a target site in a blood vessel;
causing the anchor to expand at the target site to a radially expanded position, thereby placing the electrodes into contact with a wall of the blood vessel.

12. The method of claim 11, further including, after causing the anchor to expand:
performing a mapping procedure at the target site; and
positioning a second anchor in a radially compressed position within a lumen of the anchor; and
expanding the second anchor to a radially expanded position to chronically retain the electrodes in contact with the wall of the blood vessel.

13. The method of claim 12, wherein performing a mapping procedure at the target site includes performing mapping procedures with the electrodes at one or more sites within the blood vessel, and selecting one of said sites as the target site.
